(19) United States
(12) Patent Application Publication
(10) Pub. No.: US 2007/0101453 A1
(43) Pub. Date: May 3, 2007

(54) Invention Process - Refined Cloned Super Adipose Tissue - Bio Fuel

(76) Inventor: John P. Miller, Portland, IN (US)

Correspondence Address: CONTROL GROUP CONCEPTS, 1019 WEST WALNUT STREET, PORTLAND, IN, 47371, US

(21) Appl. No.: 11/163,705

(22) Filed: Oct. 27, 2005

(51) Int. Cl. A01K 67/027

(52) U.S. Cl. 800/19

(57) ABSTRACT

Disclosed is a method of making oil byproducts off of cloned and processed fats separated into lines for different byproducts. Process can be done on a scaleable design such as a fuel tank or an old manufacturing plant and generator. Factory's make its own power off of the fuel it makes separating it into two streams, a product stream and a generator stream. It would take many of these factories to supply oil to an area but be an economical way of doing so.

Process—refined cloned super adipose tissue. The process and machine that clones super fat at a large scale and makes energy from it. In animal adipose tissue there is much fuel to be converted into bio oil products and steam. The steam in turn creates electricity. The invention a process that multiplies fat by cloning adipose tissue after it has been altered in the stages of a blast cyst (little bigger then an embryo) like all stem cells, or a straight living fat sample from the animal, then optionally converted into a super fat sample by the altering of certain genes in the sample. This same process uses a cloning tank that is scaleable in size to multiply the sample into a large workload of super fat cleaner fuel to burn, better on the environment, and until refined is not dangerous or poisonous.

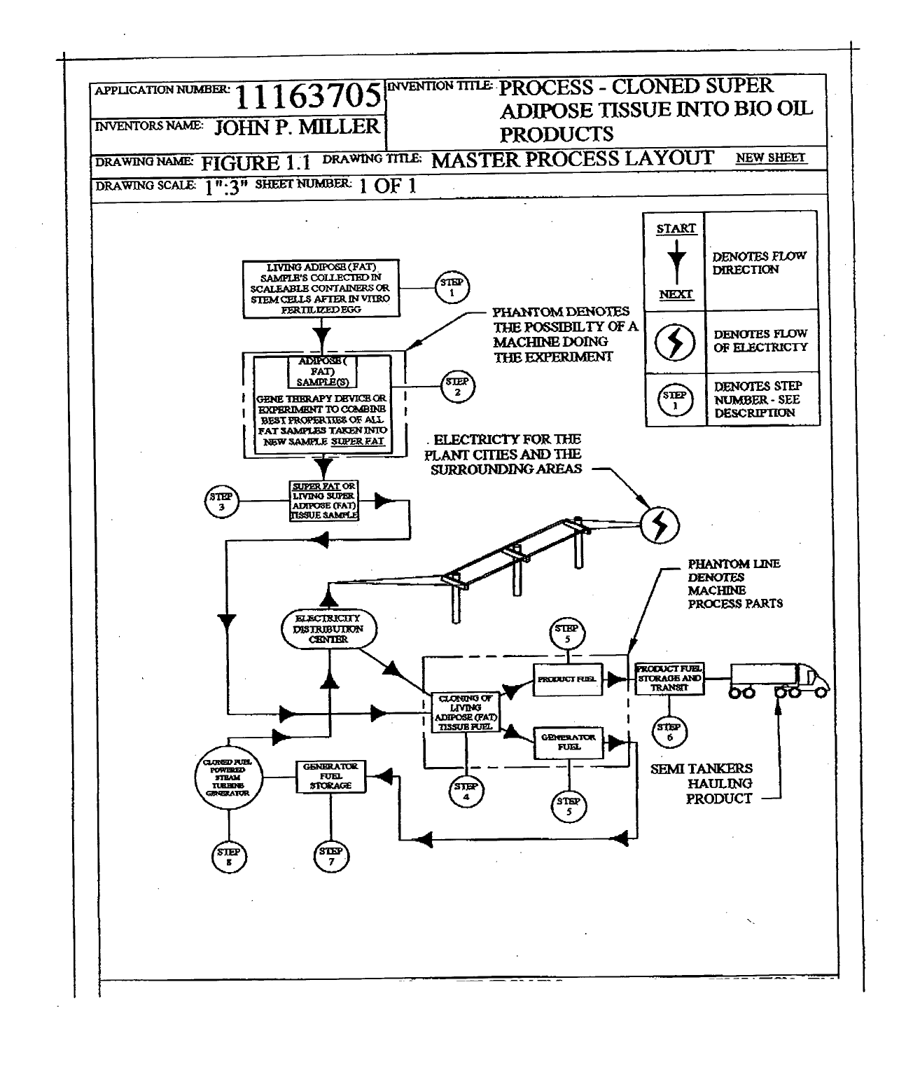

We discovered a process that makes products from cloned adipose tissue that has been processed. See FIG. 1.1 for MASTER PROCESS LAYOUT. This process does the following: One, a species of animal is found that yields the greatest amount of a fuel or oils in their adipose tissue or grown from stem cells. Two (optional), the adipose tissue is changed by method of gene therapy and then this adipose tissue is labeled super adipose tissue. Three, the adipose tissue staged into a machine, a reservoir on the machine will hold the super adipose tissue until it is ready for processing. Four, the reservoir is dumped and drained by the machine and the super adipose tissue is drained into the cloning machine. Five, the cloning machine makes scaleable amount of super adipose tissue for two sets of holding tanks. The first, set of tanks are refinement tanks for the product of fuel that make the adipose tissue into fuels. The second, set of tanks (optional) of this adipose tissue also get refined and go to the steam powered turbine generator that runs the plant and the surrounding area of cities and rural areas. Six, the refined product go to the holding tanks and then be delivered or pipelined. Seven the refined generator oil goes to the holding tanks for the generator. Eight the generator runs of refined burnable oil made from the fat. The stored product fuels are then delivered to the point of origin they are needed.

We have discovered that cloning adipose tissue after it has been altered genetically is a more efficient way of cloning adipose tissue (faty oils) and it will make better fuels with the possibility of gasoline or gasoline or diesel grade fuel. The exact match in make up to a fuel might need anatomy added to or deleted from the cell in step 2. Many samples of adipose tissue can be used to blend the best properties of the tissue that equal the best properties in the fuel in stages 6 and 7. We are after the best of the best in the each species' adipose tissue.

We have discovered designing special cloning culture will make the super adipose tissue produce more in less time. The culture we need is not of the same as standard animal blends of media and media additives to make clones or medical samples. We are relying on a vendor to spec. and or design this culture for us and our process and all the necessary chemicals to be added but water. We are asking that the average of: A. Good cloning cycle time. B. Good yield within that time.

We have discovered a machine that clones adipose tissue on a production scale to make fuels. This machine is steps 3, 4, and 5 of the process. It starts with a sample of fat sequenced into this machine or step 3. First the machine starts the fat into the sets of tanks; each tank is twice the size or bigger then the previous tank plus a working load. The working load is the amount of fat that builds up during the process that could overflow the system. Each tank drains into the next on computer controlled delays half way. We have discovered that if the cloning tanks are scaleable to meet different requirements and may even need to be modular ones to handle the amount produced efficiently. The fat is mixed in each tank with the cloning fluids through injectors in each tank. Each Tank is climate controlled, fluid controlled and constently circulating. The tanks will also have a separating system for separating the waste off the growing fat from the living tissue. As the bigger and bigger tanks fill up the great tank or the ending cloning tank dumps into a slave tank or one to hold the harvest. This fat is then rendered into product of different oils and fuels. Some of common fuel or not rendered much fuel is diverted into a separate stream the goes to a steam powered generator like ones running off of tallow.

We have discovered that refining super adipose tissues will make many types of crude oil byproducts replacements plus more. The fuels that currently could be made from a chicken fat sample are diesel. Many other current oils are made from fatlike aircraft oils. Common automobile motor oils could be made in bulk and to a higher standard.

We have discovered that if you save the super adipose tissue before it is refined it can be better for the environment, and until refined is not as dangerous as the fuels it makes.

What is claimed is:

1. We claim that oil byproducts can be made off of cloning fat separated into lines of super fat for the byproduct it makes based off a process.

2. We claim a Super Fat is made from the best qualities of different animal's fat and genes that make up a super fat blend, this blend will make a better product of oil and fuels. The fat is genetically designed through gene therapy.

3. We have discovered that cloning adipose tissue after it has been altered genetically is a more efficient way of cloning fuels and it will make better fuels.

4. We discovered a process that makes fuels from cloned adipose tissue that has been processed and refined.

5. We have discovered a machine that clones adipose tissue on a production scale to make fuels.

6. We have discovered that if the cloning tanks on the machine are scaleable to meet any requirement in the production of the byproducts.

7. We have discovered that if you save the super adipose tissue before it is refined it can be better on the environment and until refined is not as dangerous as the fuels it makes.
